(19) United States
(12) Patent Application Publication
(10) Pub. No.: US 2009/0286967 A1
(43) Pub. Date: Nov. 19, 2009

(54) Protein precipitation method and kit

(76) Inventors: Jeffrey Kitzler, Decatur, GA (US); Abdul Ibrahim, Marietta, GA (US)

Correspondence Address: Rohm & Monsanto, PLC, 12 Rathbone Place, Grosse Pointe, MI, 48230, US

(21) Appl. No.: 12/383,457

(22) Filed: Mar. 23, 2009

Related U.S. Application Data
(60) Provisional application No. 61/070,393, filed on Mar. 21, 2008.

(51) Int. Cl. C07K 1/30; C09K 3/00

(52) U.S. Cl. 530/419; 252/182.11; 252/182.32

(57) ABSTRACT

A method and kit for precipitating protein from an aqueous solution consists of a quaternary ammonium cationic surfactant and a chaotropic salt, used in combination to reversibly precipitate proteins from the aqueous solution. Precipitation separates proteins that are entrapped, non-specifically, in the quaternary ammonium cationic surfactant:chaotropic salt complex as a pellet. The precipitated protein pellet is purified further by washing with an organic solvent to remove the surfactant:chaotrope complex, leaving behind pure, concentrated proteins that can be washed to purify further the protein and/or re-dissolved in whatever buffer is most suited for any successive procedure, such as analysis.

### RELATIONSHIP TO OTHER APPLICATION

This application claims the benefit of, U.S. Provisional Application Ser. No. 61/070,393 filed on Mar. 21, 2008, the disclosure of which is incorporated herein by reference.

### BACKGROUND OF THE INVENTION

1. Field of the Invention

This invention relates generally to a method for the rapid recovery and concentration of proteins from dilute solutions, and related kit.

2. Description of the Related Art

Various methods have been developed in the art to precipitate solubilized proteins from solution, illustratively:

Trichloroaceticacid (TCA) precipitates proteins by reducing the pH to the point where the protein becomes denatured and hydrophobic patches on the proteins interact with each other leading to aggregation and falling out of solution. TCA does not precipitate all proteins quantitatively and very dilute solutions of proteins will not precipitate so that the use of a carrier protein is required. Recovery of proteins from dilute solutions can also be improved by using sodium deoxycholate in combination with TCA. Sodium deoxycholate acts as a co-precipitant in that it precipitates in an acid environment.

Ethanol causes the precipitation of proteins by lowering the dielectric constant of an aqueous solution of proteins. Ethanol interacts with the polar groups of proteins and competes with these groups for interaction with water. The proteins effectively become dehydrated, and thus, become insoluble.

Acetone also precipitates proteins by lowering the dielectric constant of the solution. A combination of TCA and acetone is used as a precipitation method to prevent the precipitation of lipids. Acetone also acts a solvent for TCA allowing for its removal at the end of the procedure.

As the ionic strength of a solution increases, it first enhances protein solubility by increasing interactions between the protein molecules and the solution. At a sufficiently high ionic strength, however, interaction with the solution becomes thermodynamically unfavorable and the protein molecules interact with each other and fall out of solution. This phenomenon is known as salting out. The concentration of salt at which this occurs varies between proteins and is a basis for protein purification or fractionation of protein solutions by precipitation. Ammonium sulfate is the salt of choice because of it is highly soluble and relatively inexpensive and has a low impact on enzyme activity.

As indicated above, Salt induced precipitation is a known method of precipitating proteins from solution. As is known in the art, protein solubility is a complex function of the physicochemical nature of the proteins, pH, temperature, and the concentration and/or type of the salt, that kosomtropic or chaotropic.

All of the foregoing methods tend to produce different results with the same starting material. Certain proteins tend to be enriched or reduced depending on the precipitation method. See, for example, Zellner, et al., Quantitative validation of different protein precipitation methods in proteome analysis of blood platelets, Electrophoresis, Vol. 26, pages 2481-2489 (2005).

Further, all of the above-described methods for protein precipitation depend upon protein-protein interactions to create the insoluble complexes. They are, thus, subject to artifacts due to differences in protein structure and/or concentration. There is, therefore, a need for a method of protein precipitation that does not rely on specific protein characteristics or concentrations.

Quaternary ammonium cationic surfactants (QACS), such as cetyltrimethylammonium bromide (CTAB), cetyltrimethylammonium chloride (CTAC), and cetylpyridinium chloride (CPC) have been used in the art to solubilize proteins. These surfactants are believed to bind to proteins in a constant detergent/protein ratio. This produces complexes with constant charge-to-mass ratios and hydrodynamic shapes irrespective of the proteins involved. This property has allowed the use of these QACS in variations of the well-known SDS-PAGE technique.

CTAB and CTAC have been used in biological research, although not for precipitating proteins. CTAB, the more widely used of the two, has been used mainly in plant nucleic acid purification. In fact, CTAB has been used to precipitate nucleic acids while leaving contaminating proteins in solution. It has also been used for preparing DNA from lambda phage and for purification of plasmid DNA from bacterial cell lysates. It has been reported that plasmid DNA precipitates over a range of 0.1% to 0.2% CTAB while leaving the contaminating protein in solution. See, Lander, et al., Fractional precipitation of plasmid DNA from lysate by CTAB, Biotechnol. Bioeng., Vol. 79, pages 776-84 (2002).

CTAC has been used to solubilize recombinant proteins from inclusion bodies in E. coli. CTAC gives a greater recovery of correctly folded protein than urea or guanidinium chloride. See, Puri, et al., Solubilization of growth hormone and other recombinant proteins from Escherichia coli inclusion bodies by using a cationic surfactant, Biochem J., Vol. 285, pages 871-879 (1992). CTAC may disrupt tertiary protein structure on binding but leaves secondary protein structure intact. As a general rule, cationic surfactants are believed to perturb protein structure much less than anionic surfactants.

As indicated above, QACS have been used to solubilize, but have not been used to precipitate proteins. However, because these surfactants bind proteins non-specifically, and at a constant ratio, they also precipitate the proteins non-specifically when they fall out of solution. QACS form micelles in aqueous solution by orienting so that their charged hydrophilic “head” groups interact with water molecules and their hydrophobic tails interact with each other. Chaotropic salts interfere with this thermodynamic state causing the surfactant micelle structure to be disrupted, leading to precipitation of the surfactant molecules. While not wishing to be bound by theory, it is believed that this property forms the basis of protein precipitation method of the present invention as will be described in detail hereinbelow. While chaotropic salts have been used to precipitate proteins, they have not been used in combination with QACS.

It is, therefore, an object of this invention to provide an improved method of protein precipitation that permits rapid recovery and concentration of proteins from dilute solutions that does not rely on specific protein characteristics or concentrations.

It is also an object of this invention to provide an improved method of protein recovery that permits rapid removal of contaminating salts and surfactants used in the precipitation process.

### SUMMARY OF THE INVENTION

The foregoing and other objects, features, and advantages are accomplished by this invention which, in one embodiment, is an improved method of protein precipitation and recovery as will be described hereinbelow.

In accordance with a method embodiment of the invention, a method for separating and recovering proteins from a sample solution comprises the steps of:
a) adding a quaternary ammonium cationic surfactant to the sample solution to form a mixture;
b) adding a chaotropic salt to the mixture to form a precipitant comprising a quaternary ammonium cationic surfactant:chaotropic salt complex with entrapped protein molecules in a supernatant liquid;
c) separating the precipitant from the supernatant liquid;
d) dispersing the precipitant in a solvent to dissolve the quaternary ammonium cationic surfactant:chaotropic salt complex; and
e) separating proteins from the dissolved quaternary ammonium cationic surfactant:chaotropic salt complex.

In the practice of this method embodiment, the quaternary ammonium cationic surfactant is selected from the group consisting of cetyltrimethylammonium bromide, cetyltrimethylammonium chloride, and cetylpyridinium chloride. In a particularly preferred embodiment of the invention, the quaternary ammonium cationic surfactant is cetylpyridinium chloride. However, cetyltrimethylammonium bromide has been used successfully in the practice of the invention.

Chaotropic salts useful in the practice of the invention include salts of the chaotropic anions iodide, perchlorate, and thiocyanate. The choice of a counterion is within the skill of a person of ordinary skill in the art. Illustratively, in the case of iodide, for example, the counterion may be sodium or potassium. In a particularly preferred embodiment, the chaotropic salt is sodium iodide.

The separating steps preferably comprise centrifuging wherein the proteins are separated from the supernatant liquid, containing the surfactant:chaotropic salt complex and/or additional contaminantes, as a pellet. In order to purify the separated proteins further, the pellet is subjected to one or more additional steps of:
f) washing the separated proteins with a further solvent to dissolve any additional salts and surfactants from the separated protein; and
g) separating the protein from the dissolved additional salts and surfactants.

The choice of solvent for the dispersing and washing steps is within the competence of a person of ordinary skill in the art. An organic solvent is used to dissolve the quaternary ammonium cationic surfactant:chaotropic salt complex to release the entrapped proteins. In preferred embodiments, the organic solvent is selected from the group consisting of acetone, acetonitrile, and mixtures of ethanol and acetonitrile, illustratively a mixture of 70:30 ethanol to acetonitrile or a 50:50 mixture. Because of the low dielectric constant of the organic solvent used in the dispersing step, the protein does not re-dissolve and remains as a precipitate. This step removes the surfactant from the protein pellet while maintaining the integrity of the protein molecules.

Organic and/or aqueous solvents can be used in the subsequent washing steps to remove additional salts and surfactants from the separated protein. Of course, the various subsequent washing steps can employ the same or different solvents. In addition to the preferred organic solvents listed above, aqueous ethanol solutions, illustratively 70% ethanol, are preferred for the subsequent washing steps. Acetonitrile may be particularly useful for the recovery of low molecular weight proteins, while 70% ethanol may be the best for removal of salt.

The purified protein may then be dissolved in any buffer that is most suitable for its further use and/or analysis.

In a second embodiment of the invention, a kit is provided for reversibly precipitating proteins from an aqueous solution. In accordance with the kit embodiment of the invention, a quaternary ammonium cationic surfactant, that may be CTAC, can be provided in liquid form as Reagent A, for example. The chaotropic salt can be provided in liquid form as Reagent B, along with instructions for use. The reagents can be provided in proper concentrations, or with instructions for dilution. The reagents can also be provided in solid form with instructions for dissolution in an appropriate solvent, which is preferably water.

In accordance with a specific illustrative embodiment, the concentration of quaternary ammonium cationic surfactant in Reagent A is in the range of 0.1%-2%, and the quaternary ammonium cationic surfactant is selected from the group consisting of CTAB, CTAC, and CPC. The quaternary ammonium cationic surfactant is preferably CTAC. In a specific illustrative embodiment, Reagent A comprises a 12.5% aqueous solution of CTAC.

The concentration of the chaotropic salt in Reagent B is between 0.025 M and 0.15 M. The chaotropic salt is preferably a salt of the chaotropic anions iodide, perchlorate, and thiocyanate, and most preferably is sodium iodide. In a specific illustrative embodiment of the invention, Reagent B is a 15% aqueous solution of sodium iodide (0.1 M).

### DETAILED DESCRIPTION

The following is a specific illustrative embodiment of the practice of the method of the present invention using a kit embodiment of the invention. As used herein, Reagent A is a QACS solution which, in this specific preferred embodiment, is a 12.5% aqueous solution of CTAC. Reagent B is a chaotropic salt, which in this specific preferred embodiment, is a 15% aqueous solution of sodium iodide.

Instructions for using the kit are provided as follows:
(1) Add 1/20 volume Reagent A to sample in a centrifuge tube and mix well.
(2) Add 1/10 volume Reagent B to the sample mixture.
(3) Allow the mixture to precipitate for about 20 minutes at room temperature to obtain a precipitate comprised of Reagent A:B complex along with the trapped protein molecules.
(4) Collect the precipitate by centrifugation and removal of the supernatant to obtain a large pellet.
(5) Completely disperse the pellet in acetone to dissolve away precipitated A:B complex. The solution should appear clear to cloudy, depending on protein concentration, with no visible clumps. Undispersed clumps will trap impurities which will be carried over into the final isolate.
(6) Collect proteins by centrifugation.
(7) Remove salts and surfactants by washing the pellet with acetone, acetonitrile or 70% ethanol. This step may be repeated, if desired, for heavily contaminated samples, or for downstream applications requiring the highest purity proteins. If necessary, collect proteins by brief centrifugation.
(8) Redissolve the pellet in the desired buffer.

The method is not selective for a particular class of protein. Reagent A binds non-specifically to proteins and the ratio of recovered proteins should reflect the proportion in the original solution. It is possible that individual proteins precipitate with slightly different efficiencies but this has not been observed in testing.

Nucleic acids do precipitate to some extent with this kit. Therefore, the kit cannot be used as a way to purify proteins away from nucleic acids, as some nucleic acid will co-precipitate.

The lower limit for reproducible recovery of BSA is 100 ng at a concentration of 0.25 μg/ml. Recovering more than 50 μg of protein in a single centrifugation tube at 200 μg/ml may not be ideal because it becomes more difficult to re-dissolve the protein pellet at the end of the procedure. Above this concentration, it may be helpful to divide the samples among several tubes or dilute the sample before precipitation.

Intact proteins in the range of 10 kD-200 kD have been precipitated successfully by the method of the instant invention as analyzed on SDS-PAGE gels.

2D electrophoresis and mass spectrometry require the sample to be as contaminant-free as possible. The further washing step, therefore, is very important when preparing a protein sample for analysis by either of these techniques. Several washes (at least 2) may be necessary to ensure that no contaminants remain with the protein pellet. In a preferred embodiment, centrifuge after each wash, being careful not to dislodge the pellet.

The choice of washing solution will depend on the application. An acetonitrile wash may help the recovery of low molecular weight proteins while 70% ethanol may be the best wash where salt removal is of utmost importance. Different washes can also be done sequentially. The best washing procedure for a given application may be determined empirically as is known to a person of ordinary skill in the art.

Most salts at concentrations used in biological laboratories will not affect the precipitation method of the present invention. However, the surrounding solution can effect kit performance in a few instances. Very high salt concentrations, e.g., a saturated solution of NaCl (5.5 M) will make it difficult to collect the pellet formed by adding reagent A and B due to the high density of the solution. In this case it may be helpful to dilute the sample before starting the precipitation. Furthermore, the presence of salts with chaotropic anions (e.g., thiocyanate, iodide, or perchlorate) will affect the performance of the kit. Solutions with these salts will cause a precipitate to form as soon as reagent A is added. Chaotropic cations (e.g., guanidine) do not have this effect. Guanidine thiocyanate will affect performance of the kit but guanidine HCI will not. Sodium iodide and sodium perchlorate will affect performance but sodium chloride will not.

The kit has been tested on protein solutions between pH 6 and pH 9 and no difference was seen in the recovery of proteins as a function of pH over this range.

In the CTAC/chaotrope system of the present invention, the added surfactant creates the precipitate, freeing the system from dependence on specific protein characteristics or concentrations. As a result, this new system is more reproducible, more universal across sample types, useful at much lower protein concentrations, and non-selective in the proteins it precipitates. In fact, better than 99% recovery of all proteins from complex mixtures has been achieved by this method. The method is capable of rapidly removing contaminating salts and surfactants, while precipitating as little as 100 ng BSA at as low as 0.25 μg/ml concentration.

Specific applications include proteomic analysis of sample fluids such as serum, saliva, urine, tears, etc. This product will enhance the assay of biological fluids that can be obtained by non-invasive means, but that are too dilute or contaminated with salt or other impurities to be analyzed directly. The method can also be used to concentrate column fractions during protein purification.

Although the invention has been described in terms of specific embodiments and applications, persons skilled in the art can, in light of this teaching, generate additional embodiments without exceeding the scope or departing from the spirit of the invention described herein. Accordingly, it is to be understood that the drawing and description in this disclosure are proffered to facilitate comprehension of the invention, and should not be construed to limit the scope thereof.

What is claimed is:

1. A method for recovering proteins from a sample solution comprising the steps of:
a) adding a quaternary ammonium cationic surfactant to the sample solution to form a mixture;
b) adding a chaotropic salt to the mixture to form a precipitant comprising a quaternary ammonium cationic surfactant:chaotropic salt complex with entrapped protein molecules in a supernatant liquid;
c) separating the precipitant from the supernatant liquid;
d) dispersing the precipitant in a solvent to dissolve the quaternary ammonium cationic surfactant:chaotropic salt complex; and
e) separating the proteins from the dissolved quaternary ammonium cationic surfactant:chaotropic salt complex.

2. The method of claim 1 further including the step of:
f) washing the separated proteins with a further solvent to dissolve any additional salts and surfactants from the separated protein; and
g) separating the protein from the dissolved additional salts and surfactants.

3. The method of claim 2 wherein steps f) and g) are repeated.

4. The method of claim 2 further comprising the step of dissolving the separated protein in a buffer.

5. The method of claim 1 wherein the quaternary ammonium cationic surfactant is selected from the group consisting of cetyltrimethylammonium bromide, cetyltrimethylammonium chloride, and cetylpyridinium chloride.

6. The method of claim 1 wherein the chaotropic salt is selected from the group consisting salts of the chaotropic anions iodide, perchlorate, and thiocyanate.

7. The method of claim 6 wherein the chaotropic salt is sodium iodide.

8. The method of claim 1 wherein the solvent is selected from the group consisting of acetone, acetonitrile, and mixtures of ethanol and acetonitrile.

9. The method of claim 2 wherein the further solvent is selected from the group consisting of acetone, acetonitrile, mixtures of ethanol and acetonitrile, and aqueous ethanol.

10. A kit for recovering proteins from a sample solution comprising:
a) a quaternary ammonium cationic surfactant Reagent A; and
b) a chaotropic salt Reagent B.

11. The kit of claim 10 wherein the concentration of quaternary ammonium cationic surfactant in Reagent A is in the range of 0.1%-2%; and the concentration of Reagent B is between 0.025 M and 0.15 M.

12. The kit of claim 10 wherein the quaternary ammonium cationic surfactant in Reagent A is selected from the group consisting of cetyltrimethylammonium bromide (CTAB), cetyltrimethylammonium chloride (CTAC), and cetylpyridinium chloride (CPC).

13. The kit of claim 12 wherein the quaternary ammonium cationic surfactant is cetyltrimethylammonium chloride.

14. The kit of claim 10 wherein the chaotropic salt in Reagent B is selected from the group consisting of salts of the chaotropic anions iodide, perchlorate, and thiocyanate.

15. The kit of claim 14 wherein the chaotropic salt is sodium iodide.
